SEQUENCE LISTING (17 sequences)

SEQ ID NO: 1 - DNA, 3425 bp, Synthetic construct
  source 1..3425: mol_type = other DNA; note = DNA Sequence of pKB0
taatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttggcatatggagctcccatggtgtacacctaggagatctgcgatcgcgtttggtctcgtgactagtgctagcggcgcgccctcgagggtaccgaattcgcggccgccggtctgataaaacagaatttgcctggcggcagtagcgcggtggtcccacctgaccccatgccgaactcagaagtgaaacgccgtagcgccgatggtagtgtgggttctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 2 - protein, 604 aa, synthetic construct
  source 1..604: note = DNA Protein Sequence of Nt.BstNBI
MAKKVNWYVSCSPRSPEKIQPELKVLANFEGSYWKGVKGYKAQEAFAKELAALPQFLGTTYKKEAAFSTRDRVAPMKTYGFVFVDEEGYLRITEAGKMLANNRRPKDVFLKQLVKWQYPSFQHKGKEYPEEEWSINPLVFVLSLLKKVGGLSKLDIAMFCLTATNNNQVDEIAEEIMQFRNEREKIKGQNKKLEFTENYFFKRFEKIYGNVGKIREGKSDSSHKSKIETKMRNARDVADATTRYFRYTGLFVARGNQLVLNPEKSDLIDEIISSSKVVKNYTRVEEFHEYYGNPSLPQFSFETKEQLLDLAHRIRDENTRLAEQLVEHFPNVKVEIQVLEDIYNSLNKKVDVETLKDVIYHAKELQLELKKKKLQADFNDPRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 3 - protein, 618 aa, synthetic construct
  source 1..618: note = Protein Sequence of Nt.BstNBI with N-terminal 6xHis tag (linker)
MGHHHHHHGSQSGGMAKKVNWYVSCSPRSPEKIQPELKVLANFEGSYWKGVKGYKAQEAFAKELAALPQFLGTTYKKEAAFSTRDRVAPMKTYGFVFVDEEGYLRITEAGKMLANNRRPKDVFLKQLVKWQYPSFQHKGKEYPEEEWSINPLVFVLSLLKKVGGLSKLDIAMFCLTATNNNQVDEIAEEIMQFRNEREKIKGQNKKLEFTENYFFKRFEKIYGNVGKIREGKSDSSHKSKIETKMRNARDVADATTRYFRYTGLFVARGNQLVLNPEKSDLIDEIISSSKVVKNYTRVEEFHEYYGNPSLPQFSFETKEQLLDLAHRIRDENTRLAEQLVEHFPNVKVEIQVLEDIYNSLNKKVDVETLKDVIYHAKELQLELKKKKLQADFNDPRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 4 - protein, 711 aa, synthetic construct
  source 1..711: note = Protein Sequence of HS5-Nt.BstNBI with N-Terminus 6xHis tag with Sumo tag
MGHHHHHHGGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQSGGMAKKVNWYVSCSPRSPEKIQPELKVLANFEGSYWKGVKGYKAQEAFAKELAALPQFLGTTYKKEAAFSTRDRVAPMKTYGFVFVDEEGYLRITEAGKMLANNRRPKDVFLKQLVKWQYPSFQHKGKEYPEEEWSINPLVFVLSLLKKVGGLSKLDIAMFCLTATNNNQVDEIAEEIMQFRNEREKIKGQNKKLEFTENYFFKRFEKIYGNVGKIREGKSDSSHKSKIETKMRNARDVADATTRYFRYTGLFVARGNQLVLNPEKSDLIDEIISSSKVVKNYTRVEEFHEYYGNPSLPQFSFETKEQLLDLAHRIRDENTRLAEQLVEHFPNVKVEIQVLEDIYNSLNKKVDVETLKDVIYHAKELQLELKKKKLQADFNDPRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 5 - protein, 735 aa, synthetic construct
  source 1..735: note = Protein Sequence of HT5-Nt.BstNBI with N-terminal 6xHis tag with a Trx tag
MGHHHHHHGSGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAENLYFQSGGMAKKVNWYVSCSPRSPEKIQPELKVLANFEGSYWKGVKGYKAQEAFAKELAALPQFLGTTYKKEAAFSTRDRVAPMKTYGFVFVDEEGYLRITEAGKMLANNRRPKDVFLKQLVKWQYPSFQHKGKEYPEEEWSINPLVFVLSLLKKVGGLSKLDIAMFCLTATNNNQVDEIAEEIMQFRNEREKIKGQNKKLEFTENYFFKRFEKIYGNVGKIREGKSDSSHKSKIETKMRNARDVADATTRYFRYTGLFVARGNQLVLNPEKSDLIDEIISSSKVVKNYTRVEEFHEYYGNPSLPQFSFETKEQLLDLAHRIRDENTRLAEQLVEHFPNVKVEIQVLEDIYNSLNKKVDVETLKDVIYHAKELQLELKKKKLQADFNDPRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 6 - protein, 678 aa, synthetic construct
  source 1..678: note = Protein Sequence of HX5-Nt.BatNBI with N-terminal 6xHis tag with a NEXT Tag
MGHHHHHHGSGSSGAVQHSNAPLIDCGAEMKKQHKEAAPEGAAPAQGKAPAAEAKKEEAPKPKPVVGIEENLYFMAKKVNWYVSCSPRSPEKIQPELKVLANFEGSYWKGVKGYKAQEAFAKELAALPQFLGTTYKKEAAFSTRDRVAPMKTYGFVFVDEEGYLRITEAGKMLANNRRPKDVFLKQLVKWQYPSFQHKGKEYPEEEWSINPLVFVLSLLKKVGGLSKLDIAMFCLTATNNNQVDEIAEEIMQFRNEREKIKGQNKKLEFTENYFFKRFEKIYGNVGKIREGKSDSSHKSKIETKMRNARDVADATTRYFRYTGLFVARGNQLVLNPEKSDLIDEIISSSKVVKNYTRVEEFHEYYGNPSLPQFSFETKEQLLDLAHRIRDENTRLAEQLVEHFPNVKVEIQVLEDIYNSLNKKVDVETLKDVIYHAKELQLELKKKKLQADFNDPRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 7 - protein, 544 aa, synthetic construct
  source 1..544: note = Protein Sequence of BsaI
MGKKAEYGQGHPIFLEYAEQIIQHKEYQGMPDLRYPDGRIQWEAPSNRKSGIFKDTNIKRRKWWEQKAISIGIDPSSNQWISKTAKLIHPTMRKPCKKCGRIMDLRYSYPTKNLIKRIRKLPYVDESFEIDSLEHILKLIKRLVLQYGDKVYDDLPKLLTCKAVKNIPRLGNDLDTWLNWIDSVYIPSEPSMLSPGAMANPPDRLDGFHSLNECCRSHADRGRWEKNLRSYTTDRRAFEYWVDGDWVAADKLMGLIRTNEQIKKETCLNDNHPGPCSADHIGPISLGFVHRPEFQLLCNSCNSAKNNRMTFSDVQHLINAENNGEEVASWYCKHIWDLRKHDVKNNENALRLSKILRDNRHTAMFILSELLKDNHYLFLSTFLGLQYAERSVSFSNIKIENHIITGQISEQPRDTKYTEEQKARRMRIGFEALKSYIEKENRNALLVINDKIIDKINEIKNILQDIPDEYKLLNEKISEQFNSEEVSDELLRDLVTHLPTKESEPANFKLARKYLQEIMEIVGDELSKMWEDERYVRQTFADLD

SEQ ID NO: 8 - protein, 618 aa, synthetic construct
  source 1..618: note = Protein Sequence of HX5-BsaI expressed with N-terminus 6xHis tag and NEXT tag
MGHHHHHHGSGSSGAVQHSNAPLIDCGAEMKKQHKEAAPEGAAPAQGKAPAAEAKKEEAPKPKPVVGIEENLYFMGKKAEYGQGHPIFLEYAEQIIQHKEYQGMPDLRYPDGRIQWEAPSNRKSGIFKDTNIKRRKWWEQKAISIGIDPSSNQWISKTAKLIHPTMRKPCKKCGRIMDLRYSYPTKNLIKRIRKLPYVDESFEIDSLEHILKLIKRLVLQYGDKVYDDLPKLLTCKAVKNIPRLGNDLDTWLNWIDSVYIPSEPSMLSPGAMANPPDRLDGFHSLNECCRSHADRGRWEKNLRSYTTDRRAFEYWVDGDWVAADKLMGLIRTNEQIKKETCLNDNHPGPCSADHIGPISLGFVHRPEFQLLCNSCNSAKNNRMTFSDVQHLINAENNGEEVASWYCKHIWDLRKHDVKNNENALRLSKILRDNRHTAMFILSELLKDNHYLFLSTFLGLQYAERSVSFSNIKIENHIITGQISEQPRDTKYTEEQKARRMRIGFEALKSYIEKENRNALLVINDKIIDKINEIKNILQDIPDEYKLLNEKISEQFNSEEVSDELLRDLVTHLPTKESEPANFKLARKYLQEIMEIVGDELSKMWEDERYVRQTFADLD

SEQ ID NO: 9 - protein, 14 aa, synthetic construct
  source 1..14: note = Protein sequence of linker 6 mer Histidine tag
MGHHHHHHGSQSGG

SEQ ID NO: 10 - protein, 107 aa, synthetic construct
  source 1..107: note = Protein sequence of Linkers and Sumo tag
MGHHHHHHGGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQSGG

SEQ ID NO: 11 - protein, 131 aa, synthetic construct
  source 1..131: note = Protein sequence of 6xHis tag with a Trx Tag
MGHHHHHHGSGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAENLYFQSGG

SEQ ID NO: 12 - protein, 78 aa, synthetic construct
  source 1..78: note = Protein sequence of 6xHis tag with a NEXT Tag
MGHHHHHHGSGSSGAVQHSNAPLIDCGAEMKKQHKEAAPEGAAPAQGKAPAAEAKKEEAPKPKPVVGIEENLYFQSGG

SEQ ID NO: 13 - protein, 111 aa, synthetic construct
  source 1..111: note = Protein sequence of eGFP one of the mutants of green fluorescent protein (GFP) from Aequorea forskalea
IDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 14 - protein, 246 aa, synthetic construct
  source 1..246: note = Protein sequence of L-eGFP is eGFP with short additional peptide at the N-terminal
GRAQSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - protein, 7 aa, synthetic construct
  source 1..7: note = Short additional peptide
GRAQSGG

SEQ ID NO: 16 - protein, 486 aa, synthetic construct
  source 1..486: note = Protein sequence of purified. HG5-eGFP is the eGFP with 6xHis and GST tag on the N-terminal
MGHHHHHHGSGSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGSGSSGENLYFQSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - protein, 247 aa, synthetic construct
  source 1..247: note = Protein sequence of 6xHis tag and GST tag
MGHHHHHHGSGSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGSGSSGENLYFQSGG

(19) United States
(12) Patent Application Publication
(10) Pub. No.: US 2025/0122489 A1
(43) Pub. Date: Apr. 17, 2025

(54) Protein Thermostability Reduction by Tag Fusion and Its Usage

(71) Applicant: Zhenyu Zhu, Lynnfield, MA (US)

(72) Inventor: Zhenyu Zhu, Lynnfield, MA (US)

(21) Appl. No.: 18/592,652

(22) Filed: Mar. 1, 2024

Related U.S. Application Data
(60) Provisional application No. 63/590,493, filed on Oct. 16, 2023.

(51) Int. Cl. C12N 9/22; C07K 14/00

(52) U.S. Cl. CPC C12N 9/22; C07K 14/001; C07K 2319/00; C07K 2319/60

(57) ABSTRACT

The invention relates to covalently bonding a protein/polypeptide with lower thermostability to a target protein with higher thermostability can reduce thermostability (increase thermolability) of the target protein. Thereafter, the target protein function can be deactivated by either heating it, even to a relatively low temperature, or by carrying out a reaction with it in the reaction mixture, at high temperature. Either way, the target protein's function is inactivated, thus reducing some of its long-acting side activity. This method for reducing thermostability of a target protein can inhibit the star activity of any restriction enzyme, including but not limited to BsaI.

### BACKGROUND

Proteins can be denatured by elevated temperature. Some functions of the protein are usually lost by denaturation. The thermostability of a protein can be changed by protein amino acids mutations, some of which can increase thermostability. For example, there are mutated and thermostable versions of T4 DNA Ligase (1), T7 RNA polymerase (2), Reverse Transcriptase (3), Lipase (4), ligninolytic oxidoreductases (5), esterase (6) and others (Note: The numbers in parenthesis refer to the numbered references in the list near the end). Other mutations are known to decrease thermostability, such as mutated Serratia marcescens nuclease (7), Proteinase K (8), human glutathione transferase P1-1 (9) and others. While thermostable mutants have maximized function even at elevated temperatures and are desirable for reactions where there are no further steps to be affected by them after their use, thermolabile mutants are desired once the reaction is completed, as they can then be deactivated by heating without interfering with subsequent reaction steps.

Fusing a tag protein or a polypeptide to a target protein is a common practice in protein expression and purification. A six-membered Histidine Tag (10) is one of the most commonly added tags and is often allowed to remain on the target protein during reaction, unless it changes the target protein function or the immune response to it. Other tags, such as GST (11), MBP (12), NusA (13), P17 (14), Sumo (15), Trx (16) and NEXT (17), are used to increase the expression level or solubility of the expressed protein. These tags are usually removed during purification by protease cleavage. In some cases, tags remain on the target protein, e.g., the tags for GST tagged TEV protease (18), and His-MBP tagged restriction endonuclease (19) are retained to facilitate removal of the enzyme by affinity resins after the reaction. For the tagged endonuclease MBP-DNaseI (20), the tag is retained for ease of production and because the tagged protein mostly retains the same function as native DNaseI.

While many proteins and enzymes are thermolabile, there are nevertheless many that retain significant function at elevated temperatures. The molecular enzymes from New England Biolabs (21) are tested for function up to 80° C. for 20 min, 95° C. for 10 min or 100° C. for 1 min. Proteins which retain function after 20 min at 80° C. are not considered heat labile. These include the following restriction endonucleases: AclI, AlwI, ApaLI, ApeKI, AveII, BamHI, BamHI-HF, BbvCI, BclI, BcoDI, BglII, BlpI, BsaAI, BsaXI, BsiEI, BsiHKAI, BsiWI-HF, BglI, BsmAI, BsrFI-v2, BssSI-v2, BstBI, BstEII-HF, BstNI, BstUI, BstYI, BstZ17I-HF, BtsI-v2, CviKI-1, CviQI, DraIII-F, Fnu4HI, FspI, HpaI, KpnI-HF, MfeI-HF, MluI-HF, MluCi, MspI, MwoI, NaeI, NciI, NgoMIV, NruI-HF, PaeR71, PpuMI, PspGI, PspXI, PstI-HF, PvuII, RsaI, SfiI, SfoI, SmlI, StuI, TaqI-v2, TfiI, TseI, Tsp45I, TspMI, TspRI, Tth111I; the nicking enzymeNb.BssSI; the homing endonuclease PI-PspI; the following DNA polymerases: Q5 Hot Start High-Fidelity DNA polymerase, Phsion Hot Start Flex DNA polymerase, One Taq Hot Start DNA polymerase, Hot Start Taq DNA polymerase, Long Amp Hot Start DNA polymerase, Hemo Klen Taq, Vent DNA polymerase, Deep Vent DNA polymerase, Sulfolobus DNA polymerase IV, Therminator DNA polymerase; the following DNA modification enzymes: T3 DNA Ligase, T7 DNA Ligase, Taq DNA Ligase, 9°N DNA Ligase, Pyrophosphases, DNaseI-XT, Thermostable FEN1, Micrococcal Nuclease, T5 exonuclease, T7 Exonuclease, T7 Endonuclease I, Tma Endonuclease III, Tth Endonuclease IV, T4 PDG, UDG, Afu UDG, BamHI Methyltransferase, HhaI Methyltransferase, MspI Methyltransferase, Taq Methyltransferase, ET SSB, Tth Argonaute; the following RNA reagents: mRNA Decapping Enzyme, Thermostable 5′ App DNA/RNA Ligase, Thermostable RnaseH, RNase HII, ShortCut RNase III; and the following glycoBiology & protein tools: Boletopsis griisea Lectin, and b-N-Acetylglucosaminidase S.

Thermo Fisher (22), includes in the descriptions of their proteins, whether they are heat sensitive or not. Their proteins described as not heat sensitive are the restriction endonucleases: BglII, Bme1390I, BseLI, BseJI, BspTI, Bst1107I, BsuRI, Cfr10I, Cfr13I, HhaI, KflI, MspI, MunI, MvaI, PsuI, PsyI, PvuII, RruI, RsaI, SdaI, SfiI, TaaI, TaiI, TaqI, TasI, TatI, TauI, Tru1I, TscAI, XmaJI; and the modification enzyme RNase I.

A fused protein with an added protein tag on the N-terminus or C-terminus of a target protein can have different thermostability than the protein tag or the target protein. While an increase in thermostability is possible in the fused protein if the thermostability of the tag is higher than the target protein, including a tag generally causes a reduction in thermostability since the thermostability of the total fused protein generally approaches the thermostability of the member (the tag or the target protein) with the lower thermostability. There are several different kinds of bases for this, firstly, the heat denatured tagged peptide or protein can block the function of the target protein by blocking its binding of substrate if the target protein is an enzyme; secondly, the denatured part can change the oligomerization status by interfere the subunit; and thirdly, the heat denatured tagged peptide or protein can precipitate from solution, thus the whole fused protein is removed from the solution and lose its function in the solution.

Different proteins or polypeptides can be fused to the target protein N-terminus. For some of them, the thermostability is similar to the target protein, while for others, the fused protein has significantly lower thermostability than the wild type, or the wild type with a six-member His tag at the C-terminus. In this way, while the target protein function generally is retained at lower reaction temperatures in the fused protein, the fused protein would be thermolabile.

The fused protein, which retains target protein function but is thermolabile, can also be used at temperatures where slow enzyme inactivation takes place. One example of this is the T5 exonuclease in the Gibson Assembly reaction (23). The T5 exonuclease chews backwards starting from the DNA end and then gradually is inactivated, allowing the polymerase to catch up to fill the gaps. A fused protein where the target protein T5 exonuclease bears a tag providing it lower thermostability, can be used in the same manner.

Many DNA restriction endonucleases have star activity, which is activity on sites other than the cognate sites. One way to reduce star activity is to change the amino acids in the endonuclease individually (24). But numerous mutants with such individual mutations must be examined to select the best one(s). The exact reason for the star activity reduction is still not clear.

Another method to reduce star activity is to use protease to control the quantity of restriction endonuclease present during the reaction (25). This method is relatively complicated and sensitive, since the concentrations of protease, restriction endonuclease and the buffer conditions need to be re-calibrated for each new reaction.

Thus, what is needed is a simple method to reduce thermostability, which can be done by covalently bonding a protein/polypeptide with lower thermostability to a target protein.

### SUMMARY

The invention relates to covalently bonding a protein/polypeptide with lower thermostability to a target protein with higher thermostability can reduce thermostability (increase thermolability) of the target protein. Thereafter, the target protein function can be deactivated by either heating it, even to a relatively low temperature, or by carrying out a reaction with it in the reaction mixture, at high temperature. Either way, the target protein's function is inactivated, thus reducing some of its long-acting side activity. One use for reducing thermostability of a target protein in such manner is to inhibit the star activity of a restriction enzyme, including but not limited to BsaI. Other restriction enzymes can be modified the same way to inhibit their star activity.

Additional aspects and advantages of the present disclosure will become apparent to those skilled in this art from the following detailed description and drawings, wherein only illustrative embodiments of the present disclosure are shown and described. The present disclosure is capable of other and different embodiments, and several details are capable of modifications in various obvious respects, and its several details are capable of modifications in various obvious respects, all without departing from the disclosure. Accordingly, the descriptions, and examples in this summary are intended for purposes of illustration only and are not intended to limit the scope of the present disclosure.

### BRIEF DESCRIPTION OF THE DRAWINGS

FIG. 1 is a restriction map for the Plasmid pKB0, showing it has one BsaI site and three distant Nt.BstNBI nicking sites.

FIGS. 2A and 2B show a comparison of the activity of Nt.BstNBI bearing different tags. Reactions were set up at 25° C., and FIGS. 2A and 2B show reaction results following gel electrophoresis of the reaction products. FIG. 2A shows the reaction results, before heating to 55° C. for 30 min. FIG. 2B shows the reaction results, after heating to 55° C. for 30 min. From the bottom to the top, FIGS. 2A and 2B display results from a: H5-Nt.BstNBI, b: HS5-Nt.BstNBI, c: HT5-Nt.BstNBI, d: HX5-Nt.BstNBI. Lanes (columns) 1 to 6, show the amount of culture in the reaction with dilutions of 1, 3, 9, 27, 81, and 243 fold. The dotted line indicates the areas of similar activity levels between panels A and B.

FIGS. 3A and 3B relate to an activity comparison (following gel electrophoresis of reaction products) of HX5-BsaI with BsaI-HFv2. FIG. 3A: the reaction was for 1 hour and in FIG. 3B the reaction was for 24 hours. From the bottom to the top of FIGS. 3A and 3B, the reagents and conditions were: a: BsaI-HFv2 at 37° C., b: HX5-BsaI at 37° C., c: BsaI-HFv2 at 50° C., d: HX5-BsaI at 50° C. Lanes 1 to lanes 12, show the amount of enzyme in the reaction with dilutions of 1, 3, 9, 27, 81, 243, 729, 2187, 6561, 19683, 59049 and 177147 fold. The “*” indicates the lane with maximum amount of restriction endonuclease which does not show star activity, and the “#” indicates the lane with minimum amount of restriction endonuclease which shows complete digestion of the substrate.

FIGS. 4A and 4B relate to showing the function and soluble protein comparison of L-eGFP and HG5-GFP.

FIG. 4A shows the fluorescence under long wavelength UV light of 100 μlexampe of 8.7 μM L-eGFP before (B2) and after (B3) 60° C. 30 min and being spun down; HG5-eGFP before (C2) and after (C3) 60° C. 30 min and being spun down.

FIG. 4B shows protein gel of 0.14 nmol of L-eGFP and HG5-GFP. Lane 1: Protein Marker, Lane 2: L-eGFP (before heating to 60° C. 30 for min and being spun down), Lane 3: L-eGFP after heating to 60° C. for 30 min and spun down. Lane 4: HG5-eGFP (before heating to 60° C. 30 for min and being spun down); Lane 5: HG5-eGFP after heating to 60° C. for 30 min and spun down.

### DETAILED DESCRIPTION

The embodiments herein and the various features and advantageous details thereof are explained more fully with reference to the non-limiting embodiments that are illustrated in the accompanying drawings and the following description. Numerous variations, changes, and substitutions may occur to those skilled in the art without departing from the invention. It should be understood that various alternatives to the embodiments of the present disclosure herein may be employed.

At the outset, for ease of reference, certain terms used in this application and their meanings as used in this context are set forth. To the extent a term used herein is not defined below, it should be given the broadest definition persons in the pertinent art have given that term as reflected in at least one printed publication or issued patent. Further, the present techniques are not limited by the usage of the terms shown below, as all equivalents, synonyms, new developments, and terms or techniques that serve the same or a similar purpose are considered to be within the scope of the present claims.

The articles “a” and “an” as used herein mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

### Making Fusion Peptides

The tags can be added to restriction enzymes by any of a number of methods, including solid phase peptide synthesis. See e.g., U.S. Pat. No. 4,764,595 (incorporated by reference). The tags can also be added by molecular cloning or polymerase chain reaction (PCR), using well-known techniques. The tags can be translated as either recombinant proteins or fusion proteins. Adding of histidine tags can be facilitated using commercial kits distributed by Qiagen (Germantown, MD).

More specifically, the tagged restriction enzymes can be expressed in any suitable host system, including a bacterial, yeast, fungal, baculovirus, plant or mammalian host cell. For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., A bacterial clone synthesizing proinsulin (1978), Proc. Natl Acad. Sci. USA 75:3727-3731), as well as the tac promoter (DeBoer et al., The tac promoter: a functional hybrid derived from the trp and lac promoters (1983), Proc. Natl Acad. Sci. USA 80:21-25).

For filamentous fungal host cells, suitable promoters for directing the transcription of the nucleic acid constructs of the present disclosure include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., Foreign gene expression in yeast: a review (1992), Yeast 8:423-488.

For baculovirus expression, insect cell lines derived from Lepidopterans (moths and butterflies), such as Spodoptera frugiperda, are used as host. Gene expression is under the control of a strong promoter, e.g., pPolh.

Plant expression vectors are based on the Ti plasmid of Agrobacterium tumefaciens, or on the tobacco mosaic virus (TMV), potato virus X, or the cowpea mosaic virus. A commonly used constitutive promoter in plant expression vectors is the cauliflower mosaic virus (CaMV) 35S promoter.

For mammalian expression, cultured mammalian cell lines such as the Chinese hamster ovary (CHO), COS, including human cell lines such as HEK and HeLa may be used to produce the tagged restriction enzyme. Examples of mammalian expression vectors include the adenoviral vectors, the pSV and the pCMV series of plasmid vectors, vaccinia and retroviral vectors, as well as baculovirus. The promoters for cytomegalovirus (CMV) and SV40 are commonly used in mammalian expression vectors to drive gene expression. Non-viral promoters, such as the elongation factor (EF)-1 promoter, are also known.

The control sequence for the expression may also be a suitable transcription terminator sequence, that is, a sequence recognized by a host cell to terminate transcription. The terminator sequence is operably linked to the 3′ terminus of the nucleic acid sequence encoding the polypeptide. Any terminator which is functional in the host cell of choice may be used.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase.

Terminators for insect, plant and mammalian host cells are also well known.

The control sequence may also be a suitable leader sequence, that is, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, Protein secretion in Bacillus species (1993), Microbiol Rev 57:109-137.

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells can be from the genes for Saccharomyces cerevisiae alpha-factor and Saccharomyces cerevisiae invertase. Signal peptides for other host cell systems are also well known.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836, incorporated by reference).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the tagged restriction enzyme relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter. Regulatory systems for other host cells are also well known.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the KRED polypeptide of the present invention would be operably linked with the regulatory sequence.

Another embodiment includes a recombinant expression vector comprising a polynucleotide encoding an engineered tagged restriction enzyme or a variant thereof, and one or more expression regulating regions such as a promoter and a terminator, and a replication origin, depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the tagged restriction enzyme at such sites. Alternatively, the nucleic acid sequences of the tagged restriction enzyme may be expressed by inserting the nucleic acid sequences or a nucleic acid construct comprising the sequences into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the tagged restriction enzyme polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector herein preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus. Selectable markers for insect, plant and mammalian cells are also well known.

The expression vectors of the present invention preferably contain an element(s) that permits integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A ori, or the origins of replication of plasmids pBR322, pUC19, pACYC177 (which plasmid has the P15A ori), or pACYC184 permitting replication in E. coli, and pUB110, pE194, pTA1060, or pAM31 permitting replication in Bacillus. Examples of origins of replication for use in a yeast host cell are the 2 micron origins of replication, ARS1, ARS4, the combination of ARS1 and CEN3, and the combination of ARS4 and CEN6. The origin of replication may be one having a mutation which makes it's functioning temperature-sensitive in the host cell (see, e.g., Ehrlich, Replication of plasmids from Staphylococcus aureus in Escherichia coli (1978), Proc Natl Acad Sci. USA 75:1433).

More than one copy of a nucleic acid sequence of the tagged restriction enzyme may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Expression vectors for the tagged restriction enzyme polynucleotide are commercially available. Suitable commercial expression vectors include p3×FLAG™ expression vectors from Sigma-Aldrich Chemicals, St. Louis Mo., which includes a CMV promoter and hGH polyadenylation site for expression in mammalian host cells and a pBR322 origin of replication and ampicillin resistance markers for amplification in E. coli. Other suitable expression vectors are pBluescriptII SK(−) and pBK-CMV, which are commercially available from Stratagene, LaJolla Calif., and plasmids which are derived from pBR322 (Gibco BRL), pUC (Gibco BRL), pREP4, pCEP4 (Invitrogen) or pPoly (Lathe et al., Plasmid and bacteriophage vectors for excision of intact inserts (1987) Gene 57:193-201.

Suitable host cells for expression of a polynucleotide encoding the tagged restriction enzyme, are well known in the art and include but are not limited to, bacterial cells, such as E. coli, Lactobacillus kefir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture mediums and growth conditions for the above-described host cells are well known in the art.

Polynucleotides for expression of the tagged restriction enzyme may be introduced into cells by various methods known in the art. Techniques include among others, electroporation, biolistic particle bombardment, liposome mediated transfection, calcium chloride transfection, and protoplast fusion. Various methods for introducing polynucleotides into cells are known to the skilled artisan.

Polynucleotides encoding the tagged restriction enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides can be prepared by chemical synthesis using, e.g., the classical phosphoramidite method described by Beaucage et al., 1981, Tet Lett 22:1859-69, or the method described by Matthes et al., Simultaneous rapid chemical synthesis of over one hundred oligonucleotides on a microscale (1984) EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., and Operon Technologies Inc., Alameda, Calif.

Engineered the tagged restriction enzyme expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B™ from Sigma-Aldrich of St. Louis Mo.

Chromatographic techniques for isolation of the tagged restriction enzyme include, among others, reverse phase chromatography high performance liquid chromatography, ion exchange chromatography, gel electrophoresis, and affinity chromatography. Conditions for purification will depend, in part, on factors such as net charge, hydrophobicity, hydrophilicity, molecular weight, molecular shape, and will be apparent to those having skill in the art.

In some embodiments, affinity techniques may be used to isolate the tagged restriction enzyme. For affinity chromatography purification, any antibody which specifically binds the tagged restriction enzyme may be used. For the production of antibodies, various host animals, including but not limited to rabbits, mice, rats, etc., may be immunized by injection with a compound. The compound may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette Guerin) and Corynebacterium parvum.

The results in the examples below show that the restriction endonuclease BsaI can be engineered into a version with much less star activity or a much higher fidelity index (26), as compared to the current HF version of the enzyme, BsaI-HFv2, available from New England Biolabs.

### EXAMPLES

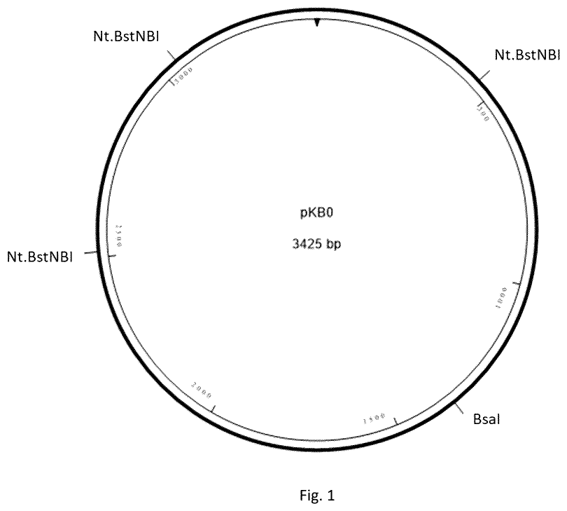

In the following two examples, the DNA substrate is a custom-made plasmid, pKB0 (SEQ ID NO: 1). The restriction endonuclease sites are shown in FIG. 1. It has one restriction endonuclease BsaI site (GGTCTCN1/N5), so it theoretically is digested into a single linear DNA. It also has three distant sites for the nicking endonuclease Nt.BstNBI (which acts at GAGTCN4\hat{ }) and whereby the Nt.BstNBI digests the plasmid into the open circular form from the supercoiled form.

This substrate selected for the testing is susceptible to the residual star activity from BsaI-HFv2 (New England Biolabs, Ipswich, MA).

### Example 1: Thermolabile Nt.BstNBI

Nt.BstNBI is a nicking endonuclease, specific for the site GAGTCN4\hat{ }, and it only nicks the top strand. When pKB0 is nicked, it changes from Supercoiled DNA to a Nicked Circular DNA form, which appears differently on agarose gel.

Nt.BstNBI (Seq ID No:2) was expressed with different tags added, i.e.: H5-Nt.BstNBI (SEQ ID NO:3 is its full sequence; the following SEQ ID NO:9 is the linker and 6-mer histidine tag: MGHHHHHHGSQSGG (SEQ ID NO:9)). HS5-Nt.BstNBI has an N-terminal 6×His tag (and linker) with a Sumo Tag (SEQ ID NO:4 is its full sequence; the following SEQ ID NO:10 is the sequence of the linkers and tags:

MGHHHHHHGGSDSEVNQEAKPEVKPEVKPETHINLKVSDGSSEIFFKIKK
TTPLRRLMEAFAKRQGKEMDSLRFLYDGIRIQADQTPEDLDMEDNDIIEA
HREQSGG (SEQ ID NO: 10)).

HT5-Nt.BstNBI has an N-terminal 6×His tag (and linker) with a Trx Tag (and linker) (SEQ ID NO: 5 is its full sequence; the following SEQ ID NO:11 is the sequence of the linkers and tags: MGHHHHHHGSGSSGSDKIIHLTDDSFDTDVLKADGAILVDFWAEWCGPCKMIAPILDEIA DEYQGKLTVAKLNIDQNPGTAPKYGIRGIPTLLLFKNGEVAATKVGALSKGQLKEFLDAN LAENLYFQSGG (SEQ ID NO:11)). HX5-Nt.BatNBI has an N-terminal 6×His tag (and linker) with a NEXT Tag (and linker) (SEQ ID NO:6 is its full sequence; the following SEQ ID NO:12 is the sequence of the linkers and tags:

MGHHHHHHGSGSSGAVQHSNAPLIDCGAEMKKQHKEAAPEGAAPAQGKAP
AAEAKKEEAPKPKPVVGIEENLYFQSGG (SEQ ID NO:12)).

Cell cultures with the expressed Nt.BstNBI were sonicated and spun down. 50 ml of the supernatant of each was incubated at 55° C. for 30 minutes. The nicking enzyme activity of the H5-Nt.BstNBI, HS5-Nt.BstNBI, HT5-Nt.BstNBI and HX5-Nt.BstNBI before and after 55° C. incubation was compared under the following conditions.
5 ml of culture, 1:3 serial dilution with water
5 ml of ZY RE Reaction Buffer 3 (50 mM Tris-HCl, 100 mM NaCl, 10 mM MgCl₂, pH 8.0)
5 ml of pKB0 at 200 ng/ml
35 ml of water

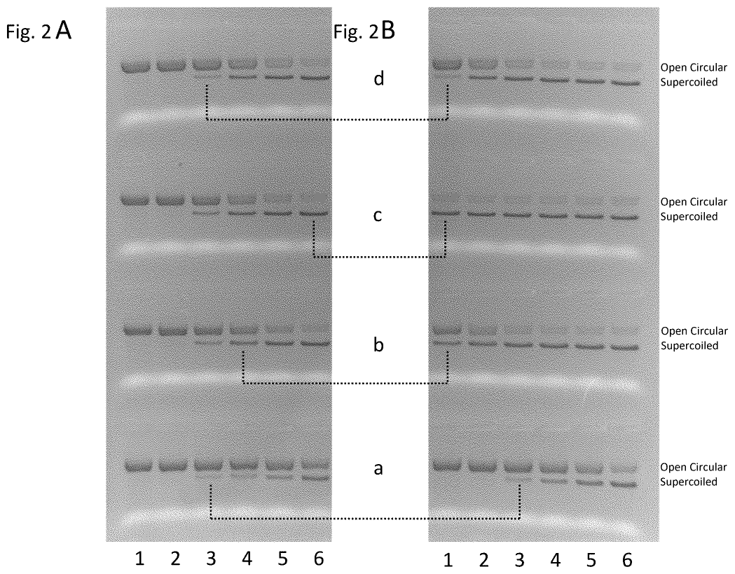

The reaction was at 25° C. for one hour. After the reaction was complete, loading dye was added to the reaction mixture and run on 0.8% Agarose gel, at 200V for 25 min (FIGS. 2A & 2B).

The results (FIG. 2A) showed that at 25° C. reaction conditions, before the 55° C. 30 min heat inactivation (FIG. 2B), H5-Nt.BstNBI, HS5-Nt.BstNBI, HT5-Nt.BstNBI, and HX5-Nt.BstNBI (FIG. 2A, rows a, b, c, d, respectively) had similar activity levels, showing that the different tags did not change the activity level much. For comparison, at dilutions of 1, 3, 9, 27, 81, and 243 times, before (FIG. 2A, columns 1 to 6, respectively) and after 55° C. for 30 min (FIG. 2B, columns 1 to 6, respectively), the activity of H5-Nt.BstNBI remained similar; HS5-Nt.BstNBI activity was reduced to about 1/27; HT5-Nt.BstNBI activity was reduced to at least about 1/243 since all lanes appeared to be inactive after the heat treatment; and HX5-Nt.BstNBI activity was reduced to about 1/9. Note that the dotted lines correlate the dilutions in FIGS. 2A and 2B where the activity in FIG. 2B, after the heat treatment, is still about the same, before the enzyme is affected.

Thus, the HT5-Nt.BstNBI is a thermolabile Nt.BstNBI variant generated through fusion with the tag protein (6×His/Trx). DNA substrate can be nicked by HT5-Nt.BstNBI at a reaction temperature, then heat inactivated at 55° C. for 30 min, then another application of the same can proceed again.

This example also suggests that fusions of tag proteins or polypeptides can greatly reduce the thermostability of the target protein. Though the proteins listed in the background section are molecular biology reagent proteins, other proteins may be chosen for fusion to reduce thermostability. Also, the tag proteins for the fusion are not restricted to those named, which assist in the production and purification. For the purpose of thermal stability reduction, all proteins with lower thermostability than the target protein (in the fusion protein), are suitable as tags to reduce thermostability.

### Example 2: High Fidelity BsaI

BsaI (SEQ ID NO:7) is a restriction endonuclease to digest double stranded DNA specific for GGTCTC (N1/N5). The wild type BsaI has star activity and had been previously engineered into BsaI-HF (24). The current commercially available version is BsaI-HFv2 (New England Biolabs, Ipswitch, MA).

HX5-BsaI (SEQ ID NO:8) was expressed with a 6×His tag and (linker)/NEXT tag at the N-terminus (SEQ ID NO:12), and purified with Nickel magnetic beads (Beaver, Suzhou, China). The protein was exchanged into storage buffer (5 mM Tris-HCl, 25 mM NaCl, 45% glycerol, pH 8.0).

A comparison of HX5-BsaI with BsaI-HFv2 were performed at their respective optimal conditions. The reaction for HX5-BsaI was set up as follows:
5 ml of HX5-BsaI, 1:3 serial diluted with diluent (10 mM Tris-HCl, 100 mg/ml BSA, pH8.0)
5 ml of ZY RE reaction buffer 3 (50 mM Tris-HCl, 100 mM NaCl, pH 8.0)
5 ml of 200 ng/ml pKB0
35 ml of water

The reaction for BsaI-HFv2 was set up as follows:
5 ml of BsaI-HFv2, specific lot in this example is 10156846, 1:3 serial diluted with diluent C (New England Biolabs, Ipswich, MA)
5 ml of Cutsmart buffer (New England Biolabs, Ipswich, MA)
5 ml of 200 ng/ml pKB0
35 ml of water

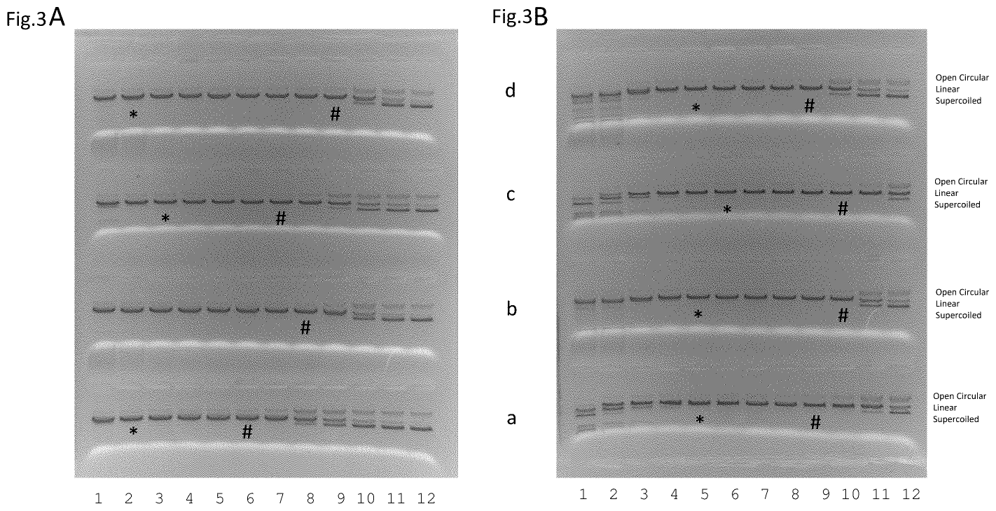

Both reactions above were performed at 37° C. and 50° C. separately, and for 1 hour and 24 hours separately. After each reaction was completed, loading dye was added to the reaction mixture and then run on 0.8% agarose gel at 200V for 25 minutes (FIGS. 3A & 3B). The reaction mixture was diluted at 1, 3, 9, 27, 81, 243, 729, 2187, 6561, 19683, 59049 and 177147 fold (columns 1 to 12, respectively, of FIGS. 3A & 3B). The * sign in FIGS. 3A & 3B indicates the dilution lane with maximum amount of restriction endonuclease which nevertheless does not show star activity. The #sign indicates the dilution lane with minimum amount of restriction endonuclease which shows complete digestion of the substrate. The difference in the dilution multiple of these two values is the fidelity index, which is set forth in Table 1:

TABLE 1
Fidelity index comparison of HX5-BsaI with BsaI-HFv2

| Conditions | HX5-BsaI | BsaI-HFv2 |
|---|---|---|
| 37° C. 1 hour | >2187 | 81 |
| 50° C. 1 hour | 2187 | 81 |
| 37° C. 24 hours | 243 | 81 |
| 50° C. 24 hours | 81 | 81 |

The fidelity index of Bsa-HFv2 remains as 81 under all conditions. While the fidelity index of HX5-BsaI lowered significantly from 1 hour to 24 hours, the lowest was at 50° C. for 24 hours, which dropped to the same value as BsaI-HFv2. The performance at 1 hour is much superior to BsaI-HFv2. Not only there is an improvement in the fidelity index, but also the star activity was reduced. The bands from star activity of BsaI-HFv2 represent the substrate being cut into much smaller fragments.

Again, while pKB0 is a specific substrate showing the potential star activity of BsaI, this result does not call into question the efficacy of the current commercial product.

The results show that instead of using internal mutations of the restriction endonucleases to alter their properties (like reducing star activity) addition of the protein tag can achieve similar or even better results. The selection of the fused tag protein is based on it having lower thermostability than the target, which can be determined through routine experimentation. Different tags for high fidelity restriction endonucleases may reduce star activity to different extents.

### Example 3: Thermo-Labile eGFP

GFP is green fluorescent protein from Aequorea forskålea (27)). It emits green fluorescence upon excitation from blue to ultraviolet light. Since sunlight has a portion of light in this spectrum, GFP appears to be green when the protein concentration is high enough.

eGFP (SEQ ID NO: 13) is one of the mutants of GFP, which is a fluorescent enhanced version. (28), it is commonly used as a model system to verify the methodology since it can be easily tracked. Here we use it as a model system to show the thermostability reduction by tagged protein fusion.

Though eGFP only can be expressed in lower temperature such as 25° C., once it is folded as a green fluorescent protein, it is very thermo-stable; especially at under 70° C. (29).

L-eGFP (Seq ID 14) is eGFP with short additional peptide, GRAQSGG, (SEQ ID NO: 15) at the N-terminal of eGFP, which is expressed in the vector pKBXD5 and from the E. Coli Strain DH10BC (ZyCloning, Woburn, MA) and subsequently purified. HG5-eGFP (SEQ ID NO: 16) is the eGFP with 6×His and GST tag on the N-terminal (SEQ ID 17) with the vector of pKBHG5 and from the E. Coli Strain DH10BC (ZyCloning, Woburn, MA) and then was subsequently purified.

50 μl L-eGFP and HG5-eGFP was incubated at 60° C. for 30 minutes. After the incubation, samples were centrifuged, and the supernatant was measured. There is not much difference of the sample of L-eGFP, A280 is changed from 0.51 to 0.40, a change of 22%. For HG5-eGFP, significant precipitation happened, and the supernatant appeared to be colorless. The absorbance changed from 2.14 to 0.16, meanwhile, the A260/A280 jumped from 0.65 to 1.70. Measured against 0.65 as a pure protein sample and 1.90 as a pure DNA sample, the remaining A280 is adjusted to 0.026. So the protein level remaining in the supernatant dropped by 98.8%.

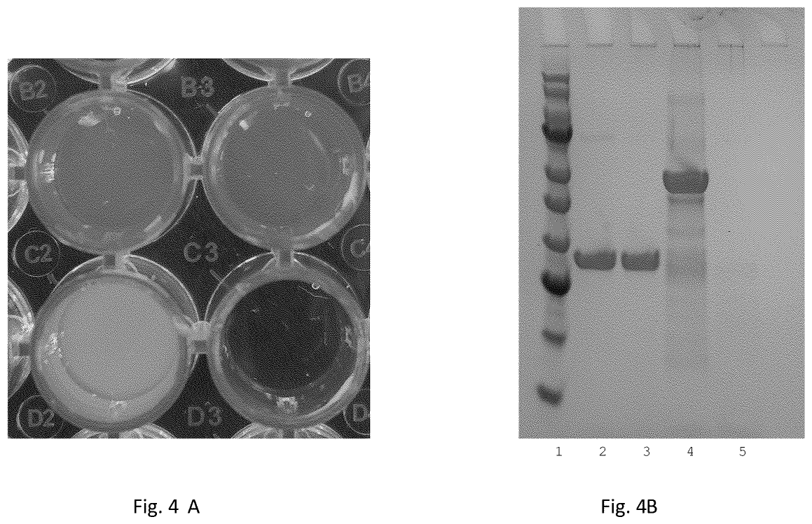

The molecular weight of L-eGFP is 27.56 kD and HG5-eGFP is 55.28 kD. The same molar starting amount of L-eGFP and HG5-eGFP were compared at fluorescence level and protein gel (FIGS. 4A&4B). The only function of eGFP is the green fluorescence. FIG. 4A showed that heating for 30 minutes at 60° C. did not affect L-eGFP much, but there was no noticeable fluorescence even in the UV light. FIG. 4B showed the protein level. While L-eGFP kept similar protein levels before and after heating for 30 min at 60° C., the major band of HG5-GFP was invisible to observation. Even in the leftover, the major band is not at the same position as the one before heat inactivation.

So, the simple conclusion from this example is that His-GST tag greatly reduced the thermostability of fused protein with eGFP. The thermostability reduction is likely because the His-GST tag has lower thermostability than the target protein. So a method to reduce a target protein thermostability, is to experimentally find proteins with lower thermostability, and then make a fusion protein of the target protein and the protein with lower thermostability. Such experimentation is routine.

### Using Tagged Restriction Enzymes

In certain embodiments, the tagged restriction enzymes are used in sequencing methods, including in attaching adapters to library fragments for subsequent sequencing. For example, in some embodiments, the tagged restriction enzymes find use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in “Applications of next-generation sequencing technologies in functional genomics” (2008), Genomics, 92:255 herein incorporated by reference.

Any number of DNA sequencing techniques are suitable, including fluorescence-based sequencing methodologies (See, e.g., Birren et al., Genome Analysis: Analyzing DNA, 1, Cold Spring Harbor, N.Y.; herein incorporated by reference). In some embodiments, the tagged restriction enzymes find uses in automated sequencing techniques understood in that art. In some embodiments, the tagged restriction enzymes finds use in parallel sequencing of partitioned amplicons (PCT Publication No: WO2006084132, herein incorporated by reference). In some embodiments, the tagged restriction enzymes finds use in DNA sequencing by parallel oligonucleotide extension (See, e.g., U.S. Pat. Nos. 5,750,341, and 6,306,597, both of which are herein incorporated by reference). Additional examples of sequencing techniques in which the tagged restriction enzymes use include the Church polony technology (Mitra et al., Fluorescent in situ sequencing on polymerase colonies (2003), Analytical Biochemistry 320, 55-65; Shendure et al., Accurate Multiplex Polony Sequencing of an Evolved Bacterial Genome (2005) Science 309, 1728-1732; U.S. Pat. Nos. 6,432,360, 6,485,944, 6,511,803; all of which are herein incorporated by reference), the 454 picotiter pyrosequencing technology (Margulies et al., Genome sequencing in microfabricated high-density picolitre reactors (2005) Nature 437, 376-380; US20050130173; herein incorporated by reference), the Solexa single base addition technology (Bennett et al., Toward the 1,000 dollars human genome. (2005) Pharmacogenomics, 6, 373-382; U.S. Pat. Nos. 6,787,308; 6,833,246; herein incorporated by reference), the Lynx massively parallel signature sequencing technology (Brenner et al. Gene expression analysis by massively parallel signature sequencing (MPSS) on microbead arrays (2000) Nat. Biotechnol. 18:630-634; U.S. Pat. Nos. 5,695,934; 5,714,330; all of which are herein incorporated by reference), and the Adessi PCR colony technology (Adessi et al. Solid phase DNA amplification: characterisation of primer attachment and amplification mechanisms (2000) Nucleic Acid Res. 28, E87; WO 00018957; herein incorporated by reference).

Next-generation sequencing (NGS) methods share the common feature of massively parallel, high-throughput strategies, with the goal of lower costs in comparison to older sequencing methods (see, e.g., Voelkerding et al., Next-Generation Sequencing: From Basic Research to Diagnostics (2009) Clinical Chem., 55:641-658; MacLean et al., Application of ‘next-generation’ sequencing technologies to microbial genetics (2009) Nature Rev. Microbiol., 7:287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLID) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., MacLean et al., supra, U.S. Pat. Nos. 6,210,891; 6,258,568; each herein incorporated by reference), template DNA is fragmented, end-repaired, ligated to adapters, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., MacLean et al., supra; U.S. Pat. Nos. 6,833,246; 7,115,400; 6,969,488; each herein incorporated by reference), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLID technology (Voelkerding et al., MacLean et al., supra U.S. Pat. Nos. 5,912,148; 6,130,073; each herein incorporated by reference) also involves fragmentation of the template, ligation to oligonucleotide adapters, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLID system, interrogation probes have 16 possible combinations of the two bases at the 3′ end of each probe, and one of four fluors at the 5′ end. Fluor color, and thus identity of each probe, corresponds to specific color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology descried herein finds use in nanopore sequencing (see, e.g., Astier et al., Toward Single Molecule DNA Sequencing: Direct Identification of Ribonucleoside and Deoxyribonucleoside 5′-Monophosphates by Using an Engineered Protein Nanopore Equipped with a Molecular Adapter, (2006) J. Am. Chem. Soc. 128 (5): 1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the tagged restriction enzymes finds use in HeliScope by Helicos BioSciences (Voelkerding et al., MacLean et al., supra; U.S. Pat. Nos. 7,169,560; 7,282,337; 7,482,120; 7,501,245; 6,818,395; 6,911,345; 7,501,245; each herein incorporated by reference). Template DNA is fragmented and polyadenylated at the 3′ end, with the final adenosine bearing a fluorescent label. Denatured polyadenylated template fragments are ligated to poly (dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., E. Pennisi, Genomics. Semiconductors inspire new sequencing technologies (2010) Science 327 (5970): 1190 U.S. Pat. Appl. Pub. Nos. 20090026082, 20090127589, 20100301398, 20100197507, 20100188073, and 20100137143, incorporated by reference). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics is used. The per-base accuracy of the Ion Torrent sequencer is ^{˜}99.6% for 50 base reads, with ^{˜}100 Mb to 100 Gb generated per run. The read-length is 100-300 base pairs. The accuracy for homopolymer repeats of 5 repeats in length is ^{˜}98%. The benefits of ion semiconductor sequencing are rapid sequencing speed and low upfront and operating costs.

The tagged restriction enzymes finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, which is incorporated by reference.

Other single molecule sequencing methods include real-time sequencing by synthesis using a VisiGen platform (Voelkerding et al., supra U.S. Pat. No. 7,329,492; U.S. patent application Ser. No. 11/671,956; U.S. patent application Ser. No. 11/781,166; each herein incorporated by reference) in which immobilized, primed DNA template is subjected to strand extension using a fluorescently-modified polymerase and florescent acceptor molecules, resulting in detectible fluorescence resonance energy transfer (FRET) upon nucleotide addition.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, including but not limited to Variant Sequences, and that such modifications and variations are within the scope of this invention as defined by the appended claims.

### Sequence Listings

SEQ ID NO: 1 DNA Sequence of pKB0
TAATGTGCCTGTCAAATGGACGAAGCAGGGATTCTGCAAACCCTATGCTACTCCGTCAAGCCGT
CAATTGTCTGATTCGTTACCAATTATGACAACTTGACGGCTACATCATTCACTTTTTCTTCACA
ACCGGCACGGAACTCGCTCGGGCTGGCCCCGGTGCATTTTTTAAATACCCGCGAGAAATAGAGT
TGATCGTCAAAACCAACATTGCGACCGACGGTGGCGATAGGCATCCGGGTGGTGCTCAAAAGCA
GCTTCGCCTGGCTGATACGTTGGTCCTCGCGCCAGCTTAAGACGCTAATCCCTAACTGCTGGCG
GAAAAGATGTGACAGACGCGACGGCGACAAGCAAACATGCTGTGCGACGCTGGCGATATCAAAA
TTGCTGTCTGCCAGGTGATCGCTGATGTACTGACAAGCCTCGCGTACCCGATTATCCATCGGTG
GATGGAGCGACTCGTTAATCGCTTCCATGCGCCGCAGTAACAATTGCTCAAGCAGATTTATCGC
CAGCAGCTCCGAATAGCGCCCTTCCCCTTGCCCGGCGTTAATGATTTGCCCAAACAGGTCGCTG
AAATGCGGCTGGTGCGCTTCATCCGGGCGAAAGAACCCCGTATTGGCAAATATTGACGGCCAGT
TAAGCCATTCATGCCAGTAGGCGCGCGGACGAAAGTAAACCCACTGGTGATACCATTCGCGAGC
CTCCGGATGACGACCGTAGTGATGAATCTCTCCTGGCGGGAACAGCAAAATATCACCCGGTCGG
CAAACAAATTCTCGTCCCTGATTTTTCACCACCCCCTGACCGCGAATGGTGAGATTGAGAATAT
AACCTTTCATTCCCAGCGGTCGGTCGATAAAAAAATCGAGATAACCGTTGGCCTCAATCGGCGT
TAAACCCGCCACCAGATGGGCATTAAACGAGTATCCCGGCAGCAGGGGATCATTTTGCGCTTCA
GCCATACTTTTCATACTCCCGCCATTCAGAGAAGAAACCAATTGTCCATATTGCATCAGACATT
GCCGTCACTGCGTCTTTTACTGGCTCTTCTCGCTAACCAAACCGGTAACCCCGCTTATTAAAAG
CATTCTGTAACAAAGCGGGACCAAAGCCATGACAAAAACGCGTAACAAAAGTGTCTATAATCAC
GGCAGAAAAGTCCACATTGATTATTTGCACGGCGTCACACTTTGCTATGCCATAGCATTTTTAT
CCATAAGATTAGCGGATCCTACCTGACGCTTTTTATCGCAACTCTCTACTGTTTCTCCATACCC
GTTTTTTTGGCATATGGAGCTCCCATGGTGTACACCTAGGAGATCTGCGATCGCGTTTGGTCTC
GTGACTAGTGCTAGCGGCGCGCCCTCGAGGGTACCGAATTCGCGGCCGCCGGTCTGATAAAACA
GAATTTGCCTGGCGGCAGTAGCGCGGTGGTCCCACCTGACCCCATGCCGAACTCAGAAGTGAAA
CGCCGTAGCGCCGATGGTAGTGTGGGTTCTCCCCATGCGAGAGTAGGGAACTGCCAGGCATCAA
ATAAAACGAAAGGCTCAGTCGAAAGACTGGGCCTTTCGTTTTATCTGTTGTTTGTCGGTGAACG
CTCTCCTGAGTAGGACAAATCCGCCGGGAGCGGATTTGAACGTTGCGAAGCAACGGCCCGGAGG
GTGGCGGGCAGGACGCCCGCCATAAACTGCCAGGCATCAAATTAAGCAGAAGGCCATCCTGACG
GATGGCCTTTTTGCGTTTCTACAAACTCTTTTGTTTATTTTTCTAAATACATTCAAATATGTAT
CCGCTCATGAGACAATAACCCTGATAAATGCTTCAATAATATTGAAAAAGGAAGAGTATGAGCC
ATATTCAACGGGAAACGTCTTGCTCGAGGCCGCGATTAAATTCCAACATGGATGCTGATTTATA
TGGGTATAAATGGGCTCGCGATAATGTCGGGCAATCAGGTGCGACAATCTATCGATTGTATGGG
AAGCCCGATGCGCCAGAGTTGTTTCTGAAACATGGCAAAGGTAGCGTTGCCAATGATGTTACAG
ATGAGATGGTCAGACTAAACTGGCTGACGGAATTTATGCCTCTTCCGACCATCAAGCATTTTAT
CCGTACTCCTGATGATGCATGGTTACTCACCACTGCGATCCCCGGGAAAACAGCATTCCAGGTA
TTAGAAGAATATCCTGATTCAGGTGAAAATATTGTTGATGCGCTGGCAGTGTTCCTGCGCCGGT
TGCATTCGATTCCTGTTTGTAATTGTCCTTTTAACAGCGATCGCGTATTTCGTCTCGCTCAGGC
GCAATCACGAATGAATAACGGTTTGGTTGATGCGAGTGATTTTGATGACGAGCGTAATGGCTGG
CCTGTTGAACAAGTCTGGAAAGAAATGCATAAGCTTTTGCCATTCTCACCGGATTCAGTCGTCA
CTCATGGTGATTTCTCACTTGATAACCTTATTTTTGACGAGGGGAAATTAATAGGTTGTATTGA
TGTTGGACGAGTCGGAATCGCAGACCGATACCAGGATCTTGCCATCCTATGGAACTGCCTCGGT
GAGTTTTCTCCTTCATTACAGAAACGGCTTTTTCAAAAATATGGTATTGATAATCCTGATATGA
ATAAATTGCAGTTTCATTTGATGCTCGATGAGTTTTTCTAATAAAAGGATCTAGGTGAAGATCC
TTTTTGATAATCTCATGACCAAAATCCCTTAACGTGAGTTTTCGTTCCACTGAGCGTCAGACCC
CGTAGAAAAGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTAGTGTAGCCGTAGTT
AGGCCACCACTTCAAGAACTCTGTAGCACCGCCTACATACCTCGCTCTGCTAATCCTGTTACCA
GTGGCTGCTGCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGGAGCGAACGAC
CTACACCGAACTGAGATACCTACAGCGTGAGCTATGAGAAAGCGCCACGCTTCCCGAAGGGAGA
AAGGCGGACAGGTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACGCGGCCTTTTTACGG
TTCCTGGCCTTTTGCTGGCCTTTTGCTCACATG
SEQ ID NO: 2: Protein Sequence of Nt.BstNBI
MAKKVNWYVSCSPRSPEKIQPELKVLANFEGSYWKGVKGYKAQEAFAKELAALPQFLGTTYKKE
AAFSTRDRVAPMKTYGFVFVDEEGYLRITEAGKMLANNRRPKDVFLKQLVKWQYPSFQHKGKEY
PEEEWSINPLVFVLSLLKKVGGLSKLDIAMFCLTATNNNQVDEIAEEIMQFRNEREKIKGQNKK
LEFTENYFFKRFEKIYGNVGKIREGKSDSSHKSKIETKMRNARDVADATTRYFRYTGLFVARGN
QLVLNPEKSDLIDEIISSSKVVKNYTRVEEFHEYYGNPSLPQFSFETKEQLLDLAHRIRDENTR
LAEQLVEHFPNVKVEIQVLEDIYNSLNKKVDVETLKDVIYHAKELQLELKKKKLQADFNDPRQL
EEVIDLLEVYHEKKNVIEEKIKARFIANKNTVFEWLTWNGFIILGNALEYKNNFVIDEELQPVT
HAAGNQPDMEIIYEDFIVLGEVTTSKGATQFKMESEPVTRHYLNKKKELEKQGVEKELYCLFIA
PEINKNTFEEFMKYNIVQNTRIIPLSLKQFNMLLMVQKKLIEKGRRLSSYDIKNLMVSLYRTTI
ECERKYTQIKAGLEETLNNWVVDKEVRF
SEQ ID NO: 3: Protein Sequence of H5-Nt.BstNBI(6xHis is single underlined)
MGHHHHHHGSQSGGMAKKVNWYVSCSPRSPEKIQPELKVLANFEGSYWKGVKGYKAQEAFAKEL
AALPQFLGTTYKKEAAFSTRDRVAPMKTYGFVFVDEEGYLRITEAGKMLANNRRPKDVFLKQLV
KWQYPSFQHKGKEYPEEEWSINPLVFVLSLLKKVGGLSKLDIAMFCLTATNNNQVDEIAEEIMQ
FRNEREKIKGQNKKLEFTENYFFKRFEKIYGNVGKIREGKSDSSHKSKIETKMRNARDVADATT
RYFRYTGLFVARGNQLVLNPEKSDLIDEIISSSKVVKNYTRVEEFHEYYGNPSLPQFSFETKEQ
LLDLAHRIRDENTRLAEQLVEHFPNVKVEIQVLEDIYNSLNKKVDVETLKDVIYHAKELQLELK
KKKLQADFNDPRQLEEVIDLLEVYHEKKNVIEEKIKARFIANKNTVFEWLTWNGFIILGNALEY
KNNFVIDEELQPVTHAAGNQPDMEIIYEDFIVLGEVTTSKGATQFKMESEPVTRHYLNKKKELE
KQGVEKELYCLFIAPEINKNTFEEFMKYNIVQNTRIIPLSLKQFNMLLMVQKKLIEKGRRLSSY
DIKNLMVSLYRTTIECERKYTQIKAGLEETLNNWVVDKEVRF
SEQ ID NO: 4: Protein Sequence of HS5-Nt.BstNBI (6xHis is single underlined, and Sumo tag is
double underlined)
MGHHHHHHGGSDSEVNQEAKPEVKPEVKPETHINLKVSDGSSEIFFKIKKTTPLRRLMEAFAKR
QGKEMDSLRELYDGIRIQADOTPEDLDMEDNDIIEAHREOSGGMAKKVNWYVSCSPRSPEKIQP
ELKVLANFEGSYWKGVKGYKAQEAFAKELAALPQFLGTTYKKEAAFSTRDRVAPMKTYGFVFVD
EEGYLRITEAGKMLANNRRPKDVFLKQLVKWQYPSFQHKGKEYPEEEWSINPLVFVLSLLKKVG
GLSKLDIAMFCLTATNNNQVDEIAEEIMQFRNEREKIKGQNKKLEFTENYFFKRFEKIYGNVGK
IREGKSDSSHKSKIETKMRNARDVADATTRYFRYTGLFVARGNQLVLNPEKSDLIDEIISSSKV
VKNYTRVEEFHEYYGNPSLPQFSFETKEQLLDLAHRIRDENTRLAEQLVEHFPNVKVEIQVLED
IYNSLNKKVDVETLKDVIYHAKELQLELKKKKLQADFNDPRQLEEVIDLLEVYHEKKNVIEEKI
KARFIANKNTVFEWLTWNGFIILGNALEYKNNFVIDEELQPVTHAAGNQPDMEIIYEDFIVLGE
VTTSKGATQFKMESEPVTRHYLNKKKELEKQGVEKELYCLFIAPEINKNTFEEFMKYNIVQNTR
IIPLSLKQFNMLLMVQKKLIEKGRRLSSYDIKNLMVSLYRTTIECERKYTQIKAGLEETLNNWV
VDKEVRF
SEQ ID NO: 5: Protein Sequence of HT5-Nt.BstNBI (6xHis tag is single underlined, and Trx tag is
double underlined)
MGHHHHHHGSGSSGSDKIIHLTDDSEDTDVLKADGAILVDEWAEWCGPCKMIAPILDEIADEYQ
GKLTVAKLNIDONPGTAPKYGIRGIPTLLLEKNGEVAATKVGALSKGOLKEELDANLAENLYFQ
SGGMAKKVNWYVSCSPRSPEKIQPELKVLANFEGSYWKGVKGYKAQEAFAKELAALPQFLGTTY
KKEAAFSTRDRVAPMKTYGFVFVDEEGYLRITEAGKMLANNRRPKDVFLKQLVKWQYPSFQHKG
KEYPEEEWSINPLVFVLSLLKKVGGLSKLDIAMFCLTATNNNQVDEIAEEIMQFRNEREKIKGQ
NKKLEFTENYFFKRFEKIYGNVGKIREGKSDSSHKSKIETKMRNARDVADATTRYFRYTGLFVA
RGNQLVLNPEKSDLIDEIISSSKVVKNYTRVEEFHEYYGNPSLPQFSFETKEQLLDLAHRIRDE
NTRLAEQLVEHFPNVKVEIQVLEDIYNSLNKKVDVETLKDVIYHAKELQLELKKKKLQADFNDP
RQLEEVIDLLEVYHEKKNVIEEKIKARFIANKNTVFEWLTWNGFIILGNALEYKNNFVIDEELQ
PVTHAAGNQPDMEIIYEDFIVLGEVTTSKGATQFKMESEPVTRHYLNKKKELEKQGVEKELYCL
FIAPEINKNTFEEFMKYNIVQNTRIIPLSLKQFNMLLMVQKKLIEKGRRLSSYDIKNLMVSLYR
TTIECERKYTQIKAGLEETLNNWVVDKEVRF
SEQ ID NO: 6: DNA Sequence of HX5-Nt.BstNBI (6xHis tag is single underlined, and NEXT tag is
double underlined)
MGHHHHHHGSGSSGAVOHSNAPLIDCGAEMKKOHKEAAPEGAAPAQGKAPAAEAKKEEAPKPKP
VVGIEENLYFMAKKVNWYVSCSPRSPEKIQPELKVLANFEGSYWKGVKGYKAQEAFAKELAALP
QFLGTTYKKEAAFSTRDRVAPMKTYGFVFVDEEGYLRITEAGKMLANNRRPKDVFLKQLVKWQY
PSFQHKGKEYPEEEWSINPLVFVLSLLKKVGGLSKLDIAMFCLTATNNNQVDEIAEEIMQFRNE
REKIKGQNKKLEFTENYFFKRFEKIYGNVGKIREGKSDSSHKSKIETKMRNARDVADATTRYFR
YTGLFVARGNQLVLNPEKSDLIDEIISSSKVVKNYTRVEEFHEYYGNPSLPQFSFETKEQLLDL
AHRIRDENTRLAEQLVEHFPNVKVEIQVLEDIYNSLNKKVDVETLKDVIYHAKELQLELKKKKL
QADFNDPRQLEEVIDLLEVYHEKKNVIEEKIKARFIANKNTVFEWLTWNGFIILGNALEYKNNF
VIDEELQPVTHAAGNQPDMEIIYEDFIVLGEVTTSKGATQFKMESEPVTRHYLNKKKELEKQGV
EKELYCLFIAPEINKNIFEEFMKYNIVQNTRIIPLSLKQFNMLLMVQKKLIEKGRRLSSYDIKN
LMVSLYRTTIECERKYTQIKAGLEETLNNWVVDKEVRF
SEQ ID NO: 7: Protein Sequence of BsaI
MGKKAEYGQGHPIFLEYAEQIIQHKEYQGMPDLRYPDGRIQWEAPSNRKSGIFKDTNIKRRKWW
EQKAISIGIDPSSNQWISKTAKLIHPTMRKPCKKCGRIMDLRYSYPTKNLIKRIRKLPYVDESF
EIDSLEHILKLIKRLVLQYGDKVYDDLPKLLTCKAVKNIPRLGNDLDTWLNWIDSVYIPSEPSM
LSPGAMANPPDRLDGFHSLNECCRSHADRGRWEKNLRSYTTDRRAFEYWVDGDWVAADKLMGLI
RTNEQIKKETCLNDNHPGPCSADHIGPISLGFVHRPEFQLLCNSCNSAKNNRMTFSDVQHLINA
ENNGEEVASWYCKHIWDLRKHDVKNNENALRLSKILRDNRHTAMFILSELLKDNHYLFLSTFLG
LQYAERSVSFSNIKIENHIITGQISEQPRDTKYTEEQKARRMRIGFEALKSYIEKENRNALLVI
NDKIIDKINEIKNILQDIPDEYKLLNEKISEQFNSEEVSDELLRDLVTHLPTKESEPANFKLAR
KYLQEIMEIVGDELSKMWEDERYVRQTFADLD
SEQ ID NO: 8: Protein Sequence of HX5-BsaI (6xHis tag is single underlined, and NEXT tag is
double underlined) (71.43 kD)
MGHHHHHHGSGSSGAVOHSNAPLIDCGAEMKKOHKEAAPEGAAPAOGKAPAAEAKKEEAPKPKP
VVGIEENLYFMGKKAEYGQGHPIFLEYAEQIIQHKEYQGMPDLRYPDGRIQWEAPSNRKSGIFK
DTNIKRRKWWEQKAISIGIDPSSNQWISKTAKLIHPTMRKPCKKCGRIMDLRYSYPTKNLIKRI
RKLPYVDESFEIDSLEHILKLIKRLVLQYGDKVYDDLPKLLTCKAVKNIPRLGNDLDTWINWID
SVYIPSEPSMLSPGAMANPPDRLDGFHSLNECCRSHADRGRWEKNLRSYTTDRRAFEYWVDGDW
VAADKLMGLIRTNEQIKKETCLNDNHPGPCSADHIGPISLGFVHRPEFQLLCNSCNSAKNNRMT
FSDVQHLINAENNGEEVASWYCKHIWDLRKHDVKNNENALRLSKILRDNRHTAMFILSELLKDN
HYLFLSTFLGLQYAERSVSFSNIKIENHIITGQISEQPRDTKYTEEQKARRMRIGFEALKSYIE
KENRNALLVINDKIIDKINEIKNILQDIPDEYKLLNEKISEQFNSEEVSDELLRDLVTHLPTKE
SEPANFKLARKYLQEIMEIVGDELSKMWEDERYVRQTFADLD
SEQ ID NO: 9: Protein sequence of linker and 6 mer Histidine
MGHHHHHHGSQSGG
SEQ ID NO: 10: Protein sequence of Linker and Sumo tag
MGHHHHHHGGSDSEVNQEAKPEVKPEVKPETHINLKVSDGSSEIFFKIKKTTPLRRLMEAFAKR
QGKEMDSLRFLYDGIRIQADQTPEDLDMEDNDIIEAHREQSGG
SEQ ID NO: 11: Protein sequence of Linker and Trx tag
MGHHHHHHGSGSSGSDKIIHLTDDSFDTDVLKADGAILVDFWAEWCGPCKMIAPILDEIADEYQ
GKLTVAKLNIDQNPGTAPKYGIRGIPTLLLFKNGEVAATKVGALSKGQLKEFLDANLAENLYFQ
SGG
SEQ ID NO: 12: Protein sequence of Linker and NEXT tag
MGHHHHHHGSGSSGAVQHSNAPLIDCGAEMKKQHKEAAPEGAAPAQGKAPAAEAKKEEAPKPKP
VVGIEENLYFQSGG
SEQ ID NO: 13: Protein sequence of eGFP
MVSKGEELFTGVVPILVELDGDVNGHKFSVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKG
IDFKEDGNILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYK
SEQ ID NO: 14: Protein sequence of L-eGFP (short linker is single underlined) (27.56 kD)
GRAQSGGMVSKGEELFTGVVPILVELDGDVNGHKFSVSGEGEGDATYGKLTLKFICTTGKLPVP
WPTLVTTLTYGVQCFSRYPDHMKQHDFFKSAMPEGYVQERTIFFKDDGNYKTRAEVKFEGDTLV
NRIELKGIDFKEDGNILGHKLEYNYNSHNVYIMADKQKNGIKVNFKIRHNIEDGSVQLADHYQQ
NTPIGDGPVLLPDNHYLSTQSALSKDPNEKRDHMVLLEFVTAAGITLGMDELYK
SEQ ID NO: 15: Short additional peptide
GRAQSGG
SEQ ID NO: 16: Protein sequence of HG5-eGFP (6xHis tag and linker is single underlined, GST tag
and linker is double underlined) (55.29 kD)
MGHHHHHHGSGSSGMSPILGYWKIKGLVOPTRLLLEYLEEKYEEHLYERDEGDKWRNKKFELGL
EFPNLPYYIDGDVKLTOSMAIIRYIADKHNMLGGCPKERAEISMLEGAVLDIRYGVSRIAYSKD
FETLKVDFLSKLPEMLKMFEDRLCHKTYLNGDHVTHPDEMLYDALDVVLYMDPMCLDAFPKLVC
FKKRIEAIPOIDKYLKSSKYIAWPLOGWOATFGGGDHPPKGSGSSGENLYFOSGGMVSKGEELF
TGVVPILVELDGDVNGHKFSVSGEGEGDATYGKLTLKFICTTGKLPVPWPTLVTTLTYGVQCES
RYPDHMKQHDFFKSAMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNTPIGDGPVLLPDNHY
LSTQSALSKDPNEKRDHMVLLEFVTAAGITLGMDELYK
SEQ ID NO: 17: Protein sequence of 6xHis tag and GST tag
MGHHHHHHGSGSSGMSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVLDIRYGVSRIAYSKD
FETLKVDFLSKLPEMLKMFEDRLCHKTYLNGDHVTHPDFMLYDALDVVLYMDPMCLDAFPKLVC
FKKRIEAIPQIDKYLKSSKYIAWPLQGWQATFGGGDHPPKGSGSSGENLYFQSGG

### REFERENCES (THOSE IDENTIFIED BY NUMBER IN THE SPECIFICATION)

1) Ong, J. (2020) U.S. Pat. No. 10,837,009B1, DNA ligase variants
2) Boulain J.-C. et al., (2013) Mutants with higher stability and specific activity from a single thermosensitive variant of T7 RNA polymerase, Protein Eng Des Sel 26 (11): 725-34. doi: 10.1093/protein/gzt040.
3) Mohr, S. et al., (2013) Thermostable group II intron reverse transcriptase fusion proteins and their use in cDNA synthesis and next-generation RNA sequencing. RNA, 19 (7): 958-70. doi: 10.1261/rna.039743.113.
4) Yoshida, K., et al., (2021) Enhancement of protein thermostability by three consecutive mutations using loop-walking method and machine learning. Sci Rep 11, 11883
5) García-Ruiz, E. et al. (2010) Evolving thermostability in mutant libraries of ligninolytic oxidoreductases expressed in yeast. Microb Cell Fact 9, 17
6) Giver, L., et al. (1998) Directed evolution of a thermostable esterase. PNAS 95 (22) 12809-12813
7) Zhu, Z., et al., (2021) U.S. Pat. No. 10,920,202B1 Thermolabile Serratia Marcescens nuclease
8) Chen, M., et al., (2020) U.S. Pat. No. 10,633,644B1 Proteinases with improved properties
9) Rossjohn, J., et al., (2000) Structures of thermolabile mutants of human glutathione transferase P1-1 JMB, Volume 302, Issue 2, 15, Pages 295-302
10) Hochuli E et al. (1988). Genetic Approach to Facilitate Purification of Recombinant Proteins with a Novel Metal Chelate Adsorbent. Bio/Technology. 6 (11): 1321-5.
11) Sandra H et al. (2011) Purification of proteins fused to glutathione S-transferase Methods Mol Biol. 2011; 681:259-280
12) di Guan C. et al. (1988). Vectors that facilitate the expression and purification of foreign peptides in Escherichia coli by fusion to maltose-binding protein. Gene. 67 (1): 21-30.
13) Roger G. (1999) U.S. Pat. No. 5,989,868A, Fusion protein systems designed to increase soluble cytoplasmic expression of heterologous proteins in Escherichia coli
14) Wang, Y., et al. (2022) A 33-residue peptide tag increases solubility and stability of Escherichia coli produced single-chain antibody fragments. Nat Commun 13, 4614.
15) Christopher D. et al. (2008) U.S. Pat. No. 7,910,364B2, Rapidly cleavable sumo fusion protein expression system for difficult to express proteins
16) Hisabori, T. et al. (2005) Thioredoxin affinity chromatography: a useful method for further understanding the thioredoxin network Journal of Experimental Botany, Volume 56, Issue 416, Pages 1463-1468
17) Jo, B. H. (2022) An Intrinsically Disordered Peptide Tag that Confers an Unusual Solubility to Aggregation-Prone Proteins, Appl Environ Microbiol. 88 (7): e00097-22.
18) Blommel P. G. (2007) A Combined Approach to Improving Large-Scale Production of Tobacco Etch Virus Protease, Protein Expr Purif.; 55 (1): 53-68.
19) Zhu; Zhenyu et al. (2023) U.S. Pat. No. 11,767,524B2, His-MBP tagged DNA endonuclease for facilitated enzyme removal
20) https://www.neb.com/en-us/products/m0303-dnase-i-rnase-free#Product%20Information Product%20Notes
21) https://www.neb.com
22) https://www.thermofisher.com/search/browse/category/us/en/90226288?query=Sensitive%20 to%20Heat%20Inactivation%09No&focusarea=Restriction%20Enzymes
23) Gibson D G et al. (2009). Enzymatic assembly of DNA molecules up to several hundred kilobases. Nature Methods. 6 (5): 343-345.
24) Zhu, Z., et al, (2008) U.S. Pat. No. 8,372,619B2 High fidelity restriction endonucleases
25) Zhu, Z., et al, (2022) U.S. Pat. No. 11,512,296B2 Using proteases to control restriction enzyme activity
26) Wei, H. et al., (2008) The Fidelity Index provides a systematic quantitation of star activity of DNA restriction endonucleases. Nucleic Acids Res. 2008 May; 36 (9): e50. doi: 10.1093/nar/gkn182.
27) Prendergast, F G. et al. (1978) Chemical and physical properties of aequorin and the green fluorescent protein isolated from Aequorea forskålea. Biochemistry. 17 (17): 3448-53.
28) Cinelli, R A. et al., (2000) The enhanced green fluorescent protein as a tool for the analysis of protein dynamics and localization: local fluorescence study at the single-molecule level. Photochem Photobiol, 71 (6): 771-6
29) Nagy, A. et al., (2004) Thermal stability of chemically denatured green fluorescent protein (GFP): A preliminary study. Thermochemica Acta, 410 (1-2), 161-163.

What is claimed is:

1. A method to reduce the thermostability of a target protein, comprising:
fusing a protein or polypeptide with less thermostability than the target protein to the N-terminus or C-terminus of the target protein, which results in a fusion product with lower thermostability from that of the target protein.

2. The method of claim 1 wherein the target protein is Nt.BstNBI and the other protein has the sequence of SEQ ID NO: 11 and is fused to the target protein N-terminus.

3. The method to create high fidelity restriction endonuclease by fusing a protein or polypeptide with less thermostability than the restriction endonuclease to the N-terminus or C-terminus of a restriction endonuclease.

4. The method of claim 1 wherein the target protein is BsaI and the other protein has the sequence of SEQ ID NO:12 and is fused to the target protein N-terminus.

5. The method of claim 1 wherein the target protein is eGFP and the other protein has the sequence of SEQ ID NO:17 and is fused to the target protein N-terminus.
